(19) United States
(12) Patent Application Publication
(10) Pub. No.: US 2026/0096761 A1
(43) Pub. Date: Apr. 9, 2026

(54) SYSTEM FOR QUANTIFICATION OF HUMAN TEMPOROMANDIBULAR JOINT MECHANICS AND ORAL FUNCTION

(71) Applicant: Clemson University, Clemson, SC (US)

(72) Inventors: Brooke Damon, Summerville, SC (US); Shuchun Sun, Charleston, SC (US); Hai Yao, Mount Pleasant, SC (US); Janice Lee, Bethesda, MD (US)

(73) Assignee: Clemson University, Clemson, SC (US)

(21) Appl. No.: 19/349,807

(22) Filed: Oct. 3, 2025

Related U.S. Application Data
(60) Provisional application No. 63/702,999, filed on Oct. 3, 2024.

(51) Int. Cl. A61B 5/22; A61B 5/00; A61B 5/11; A61B 5/389; A61B 7/00; A61B 90/00

(52) U.S. Cl. CPC A61B 5/228; A61B 5/0077; A61B 5/1114; A61B 5/1127; A61B 5/389; A61B 5/4542; A61B 5/4824; A61B 5/7425; A61B 7/006; A61B 90/39; A61B 2090/064; A61B 2090/3937; A61B 2090/3966; A61B 2090/3995

(57) ABSTRACT

A system having a skull tracking bracket; a mandible motion tracking bracket; a motion detecting camera; an electromyography sensors adapted to detect and measure facial muscle activation data and transmit the data to the computerized controller; an audio sensor carried by the subject and adapted to detect and measure sound data associated with jaw (and joint) movement data and transmit the data to the computerized controller; a bite force sensor adapted to detect and measure static and dynamic bite force data; a pain push-button switch (or other sensor) adapted to be activated by the subject when experiencing pain; and a computerized controller is adapted to receive motion data, facial muscle data, joint sound data, static and dynamic bite force data, pain data, and received a set of three-dimensional images of the subject, overlay the detected motion with the three-dimensional image and display the overlay.

### RELATED APPLICATION

This application claims priority to U.S. Provisional Application No. 63/702,999 filed Oct. 3, 2024, which is hereby incorporated by reference in its entirety.

### GOVERNMENT LICENSE RIGHTS

This invention was made with government support under R01DE021134 (PPN: 2020001633) and U01DE031512 (PPN: 2021001802) awarded by NIH. The United States government has certain rights in the invention.

### BACKGROUND OF THE INVENTION

### 1) Field of the Invention

This system is an integrated temporomandibular joint (TMJ) and oral functional assessment tool for chairside evaluation of human masticatory function and TMJ biomechanics during oral tasks, utilizing a combination of novel cone-beam computed tomography (CBCT) co-registered 3D jaw motion tracking, surface electromyography (sEMG), bite force measurements, pain recording, and joint clicking sound recording.

### 2) Description of the Related Art

TMJ is essential for daily functions such as chewing and speaking, but it is susceptible to pain and dysfunctions, known as temporomandibular disorders (TMD). Understanding the relationships between TMJ structure, function, and pain is essential for understanding TMD etiology. Existing functional assessment tools lack the ability to simultaneously record pain and are difficult to integrate, making them challenging to implement in dental clinics.

Humans have two TMJs. The THJ connect condyles of the mandible to each side of the fossa of the skull and help with movements like chewing and speaking.

The temporomandibular joint (TMJ) is essential for daily activities such as chewing and speaking. These tasks require the TMJ to perform an extensive set of complex movements while bearing significant mechanical loads, making the joint and its associated structures particularly susceptible to pain and dysfunction. Musculoskeletal conditions characterized by pain and/or dysfunction in the TMJ, masticatory muscles, and related structures are collectively known as TMJ disorders (TMD). TMD, which affect 4-12% of the general population and lead 3-10% to seek clinical treatment, lower quality of life for the affected population and place a heavy burden on healthcare costs. The etiology of TMD is multifactorial, with factors such as altered structure and abnormal function playing crucial roles. Structural changes in the TMJ are highly prevalent in TMD patients with condylar flattening and articular surface erosion being the most commonly observed changes. However, the fact that some patients with painful TMD do not exhibit degenerative changes in the joint, while others with degenerative changes maintain proper function without pain, indicates that structural changes are only part of the picture. Besides structure, functional factors also play a crucial role in TMD. Abnormalities in jaw movement, joint mechanics and masticatory muscle coordination are not only commonly observed in TMD patients but also contribute to TMD development, often manifesting as pain or loss of function during activities like mouth opening or chewing. Structure provides the basis for normal function, and abnormalities in function can lead to TMJ degenerative changes and pain; dysfunctional interactions between structure and function can result in painful TMD. The complex interactions between TMJ structure, function, and pain underscore the need for a comprehensive approach that considers all three aspects in the patient. To achieve this comprehensive approach, it is crucial for dental clinics to have the capacity to simultaneously gather structural, functional, and pain data from patients at the dental chairside.

TMJ dysfunctions are conditions affecting jaw joints and surrounding muscles and ligaments. TMD can result in jaw pain, headaches and difficulty opening and closing the mouth. It is estimated that between 5% and 12% of the general adult population have some form of TMJ disorder.

Categories of TMDs include disorders of the joints; disorders of the associated muscles; and headaches and pain that result from TMD. However, chronic TMD is usually a multisystem disorder with overlapping co-morbidity. Clinical efficacy requires isolation of findings that have prognostic or etiological significance. There is a need for a system that will assist dental and musculoskeletal researchers, otolaryngologists, orthognathic surgeons, prosthodontists, and orthodontists in analyzing, diagnosing, preventing, and modeling TMD, and providing TMD correction and prosthetics.

It would be advantageous to have a system which combined CBCT co-registered 3D jaw motion tracking, sEMG, bite force measurements, pain recording, joint clicking sound recording and some combination of these to provide for quantification of 3D TMJ biomechanics.

It would be advantageous for a system to capture human masticatory function and TMJ biomechanics data and provide such data as input for novel software and computational modeling, provide baseline masticatory function and aid clinicians in diagnosing TMJ disorders as well as quantify treatment efficacy.

### SUMMARY OF THE INVENTION

The above objectives are accomplished by providing a system for quantification of human temporomandibular joint mechanics and oral function comprising a skull motion tracking bracket configured to be carried by a subject and having a plurality of motion tracking markers positioned asymmetrically thereon, a mandible motion tracking bracket configured to be carried by the subject and having a plurality of motion tracking markers positioned thereon, a set of motion detecting cameras adapted to detect and record data representing movement of the motion tracking markers on the skull motion tracking bracket and the mandible motion tracking bracket, and a computerized controller adapted to receive the motion data from the set of motion detecting cameras, receive a set of three-dimensional images of the subject, co-register the motion data with the three-dimensional images, and display an overlay of the detected motion with the three-dimensional images to a user.

Current systems in dental clinics can only address one or two aspects at a time, making it difficult to simultaneously evaluate structure, function, and pain, understand their relationships, and develop more effective diagnostic and treatment plans. Existing functional assessment tools lack the ability to simultaneously record pain and are difficult to integrate, making them challenging to implement in dental clinics. Systems such as Planmeca and Sicat JMT+ capture TMJ movement by augmenting CBCT machines, but they are limited in their ability to evaluate multiple oral tasks across several sessions due to the use of ionizing radiation. Other systems use techniques like magnets, infrared sensors, or optical cameras to track trajectories of markers attached to the mandible or skull, but none of these systems can simultaneously record function and pain. These systems are also difficult to integrate for assessing multiple TMJ functions, as different vendors use proprietary hardware and software that are not easily compatible, hindering the accuracy and efficiency of TMJ functional assessments.

The system may include a set of electromyography sensors carried by the subject and adapted to detect and record facial muscle activity data and transmit the data to the computerized controller. The system may include a set of audio sensors carried by the subject and adapted to detect and record sound data associated with jaw movement and transmit the data to the computerized controller. The system may include a bite force sensor adapted to detect and record bite force data of the subject and transmit the bite force data to the computerized controller. The system may include a pain actuator adapted to be activated by the subject to indicate pain and transmit pain data to the computerized controller.

The skull motion tracking bracket may comprise at least seven motion tracking markers and the mandible motion tracking bracket may comprise at least six motion tracking markers. The motion tracking markers may comprise retroreflective microspheres attached to a surface of a sphere containing cone-beam computed tomography contrast material.

According to another aspect of the present disclosure, a system for quantification of human temporomandibular joint mechanics and oral function is provided. The system comprises a skull motion tracking bracket configured to be carried by a subject and having motion tracking markers positioned thereon, a mandible motion tracking bracket configured to be carried by the subject and having motion tracking markers positioned thereon, a set of motion detecting cameras adapted to detect and record motion data from the motion tracking markers, a set of electromyography sensors configured to be carried by the subject and adapted to detect and record facial muscle activity data, a set of audio sensors configured to be carried by the subject and adapted to detect and record sound data associated with jaw movement, a bite force sensor adapted to detect and record bite force data of the subject, and a computerized controller adapted to receive the motion data, the facial muscle activity data, the sound data, and the bite force data and synchronize the received data for analysis.

The system may include a pain actuator configured to detect a pain signal from the subject and wherein the computerized controller is adapted to receive the pain signal and synchronize the received data for analysis. The skull motion tracking bracket may comprise a plurality of motion tracking markers positioned asymmetrically thereon and the mandible motion tracking bracket may comprise a plurality of motion tracking markers positioned thereon. The motion tracking markers may comprise retroreflective microspheres attached to a surface of a sphere containing cone-beam computed tomography contrast material.

The bite force sensor may be configured to operate in both static and dynamic bite modes, the static mode measuring bite force at a nearly closed mouth position and the dynamic mode measuring bite force during mandibular motion under load. The bite force sensor may comprise a piezoresistive force sensor within a flexible bite surface cover and may be driven by an operational amplifier circuit to create an analog voltage signal. The set of electromyography sensors may comprise surface electrodes configured to be placed on left and right masseter and temporalis muscles of the subject. The set of audio sensors may comprise a highly sensitive MEMS microphone within an acoustic chamber configured to be placed directly on a temporomandibular joint area of the subject.

According to another aspect of the present disclosure, a temporomandibular joint assessment apparatus is provided. The apparatus comprises a skull tracking bracket having retroreflective motion tracking markers visible in cone-beam computed tomography imaging, a mandible tracking bracket having retroreflective motion tracking markers and configured to attach to anterior mandibular teeth, infrared motion tracking cameras positioned to capture three-dimensional motion of the retroreflective motion tracking markers, a bite force measurement device having static and dynamic measurement modes and including a pain indicator button, electromyography sensors configured for placement on masticatory muscles, a microphone configured to detect temporomandibular joint clicking sounds, and a data collection hub configured to synchronize and record data from the infrared motion tracking cameras, the bite force measurement device, the electromyography sensors, and the microphone at a sampling rate of at least 1000 Hz.

The skull tracking bracket may comprise a plurality of seven retroreflective motion tracking markers positioned thereon. The mandible tracking bracket may comprise a plurality of retroreflective motion tracking markers positioned thereon. The bite force measurement device may comprise a piezoresistive force sensor and may be driven by an operational amplifier circuit to create an analog voltage signal. The bite force measurement device in dynamic measurement mode may comprise two hinged bite surfaces loaded via a torsional spring to allow measurement of bite force during mandibular motion under load.

### BRIEF DESCRIPTION OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1A is a flow chart showing aspects of the system.

FIG. 1B is a flow chart showing aspects of the system.

FIG. 1C shows images of aspects of the hardware of this system.

FIG. 1D shows a user screen that is provided by the system.

FIG. 1E shows images of aspects of the hardware of this system.

FIG. 1F shows images of aspects of the hardware of this system.

FIG. 1G shows images of aspects of the hardware of this system.

FIG. 2 shows aspects of the hardware and a subject with the skull tracking bracket and mandible motion tracking bracket in position.

FIG. 3 shows a bite force device with progressive read out.

FIG. 4 demonstrates the use of the bite force device with mandible motion tracking devices.

FIG. 5 shows tracking markers 502 isolated from the skull tracking bracket and mandible motion tracking bracket and as positioned relative to the maxilla and mandible.

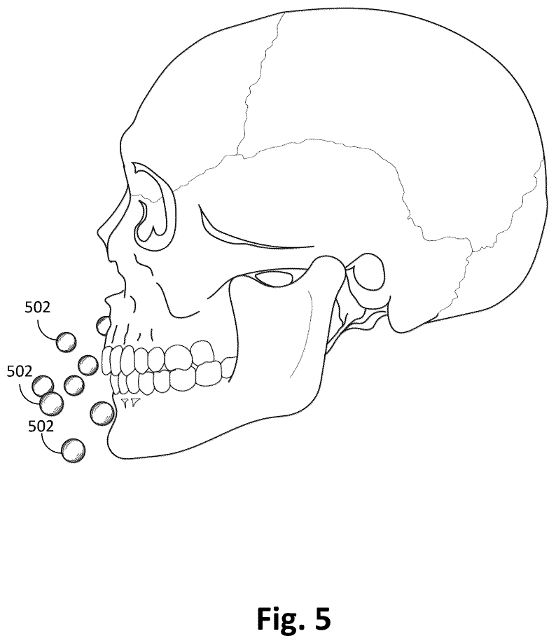

While each of the drawing figures depicts a particular embodiment for purposes of depicting a clear example, other embodiments may omit, add to, reorder, and/or modify any of the elements shown in the drawing figures. For purposes of depicting clear examples, one or more figures may be described with reference to one or more other figures, but using the particular arrangement depicted in the one or more other figures is not required in other embodiments. The drawings and schematic representations are intended to support the understanding of the invention. These may not be to scale and are not intended to limit the invention to any particular layout, connectivity, or architectural implementation. Correspondence between drawing elements and described components is provided for illustrative purposes and should not be interpreted to limit the claim scope.

### DETAILED DESCRIPTION OF EMBODIMENTS OF THE INVENTION

The present system is an innovative system and method for the collection and analysis of masticatory function and TMJ biomechanics, including jaw kinematics, muscle activation, audible joint effects and bite force data, during oral tasks. The invention provides devices for mandible motion tracking and recording using a skull tracking bracket and mandible motion tracking bracket having tracking markers. Data collected from the devices may be used for patient diagnostics and treatment, and for building state-of-the-art multiscale biomechanical models to analyze the mechanical and biological environment inside the jaw and jaw joints, as well as providing a tool for studying jaw disease etiology.

This system improves the current dental equipment of the dental office. Currently, equipment in dental clinics can only address one or two aspects at a time, making it difficult to simultaneously evaluate structure, function, and pain, understand their relationships, and develop more effective diagnostic and treatment plans. In dental clinics, TMJ structures can be examined using imaging techniques such as cone beam computed tomography (CBCT) and magnetic resonance imaging (MRI), but functional assessment and pain recording tools are still in need of further development. TMJ functional assessment ultimately boils down to evaluating joint movement, masticatory muscle function and joint load during oral tasks. Currently equipment focuses on one aspect such as the Planmeca and Sicat JMT+ which capture TMJ movement by augmenting CBCT machines, but they are limited in their ability to evaluate multiple oral tasks across several sessions due to the use of ionizing radiation.

Other systems use techniques like magnets, infrared sensors, or optical cameras to track the trajectories of markers attached to the mandible or skull. The captured trajectories are then registered to the bone geometry obtained from CBCT to accurately map jaw motion. Systems like 4D Motion Capture (Modjaw) offer functionalities such as real-time motion preview and additional analysis modules. The JMA Optics System can be enhanced with the JMA Analyzer to provide electromyography (EMG) measurement functionality, enabling analysis of muscle activation. Customized jaw motion tracking systems have also been developed in research labs, primarily aimed at advancing scientific research. Emerging motion capture systems using accelerometry are being developed in research labs, with the potential for future chairside or even take-home applications. Masticatory muscle function is typically evaluated through palpation, and EMG measurement systems are commercially available. Additionally, vendors such as Tekscan provide bite force measurement systems, which are critical for evaluating occlusal function, supplementing the assessment of joint function.

However, none of these systems can simultaneously record function and pain. Simultaneously capturing both function and pain is critical, as it allows researchers and surgeons to trace pain back to corresponding functional parameters, such as jaw motion, muscle activation, and joint load, enabling a better understanding of how the interaction between structure and function contributes to pain.

Besides lacking the critical pain recording component, these systems are also difficult to integrate for assessing multiple TMJ functions, as different vendors use proprietary hardware and software that are not easily compatible, hindering the accuracy and efficiency of TMJ functional assessments. Performing multiple functional assessments together is essential for collecting the comprehensive data needed for various applications. For example, calculating muscle and joint forces using computational models requires multiple accurate data from functional assessments, including jaw kinematics, masticatory muscle activation, and bite force during oral tasks. When combined with morphological information from CBCT and MRI, these functional data enable accurate estimations of joint load and masticatory muscle force. In a dental clinic, where time is limited and patient flow is high, efficiency and ease of use are critical for the adoption of a functional assessment system. The use of multiple software and hardware systems not only complicates the process but also adds significant burdens to clinical staff, increasing the risk of errors and reducing productivity. An integrated, user-friendly TMJ assessment system capable of seamlessly collecting multimodal data would not only reduce staff workload but also facilitate efficient and accurate functional assessment, significantly improving clinical outcomes and streamlining patient care.

To address these challenges of the current technology and improve the understanding of the relationships between TMJ structure, function, and pain in both TMD research and clinical practice, this system integrates TMJ functional assessment system with a user-friendly interface, capable of evaluating multiple biomechanical functions and equipped with features for recording pain timing. In one embodiment, the system can be applied for dental chairside TMJ functional assessment where one embodiment has been tested at the National Institutes of Health (NIH) clinical center, where clinical staff have successfully assessed 42 patients with craniofacial deformities, demonstrating its efficacy and ease of use.

Obtaining real patient data assisted in understanding the complex and multifactorial etiology of TMD and can be used for improving clinical prognosis, diagnosis, and post-treatment evaluation. This system enables the efficient collection of multi-modal data to study the relationship between TMJ structure, function, and pain utilizing advanced data-driven approaches like machine learning. This capability enhances clinical practice by allowing for comprehensive, quantitative assessments of TMJ function, leading to more accurate diagnoses and personalized treatment plans. Additionally, the adaptable nature of this system's concept extends its potential application to other musculoskeletal disorders, further broadening its clinical impact.

The invention provides quantification of human masticatory function and TMJ biomechanics. In one embodiment, the invention uses a combination of CBCT, co-registered 3D jaw motion tracking, sEMG, and bite force measurements. The system includes hardware for recording motion, electromyograph (EMG), bite force, pain and joint clicking sound, and includes user-friendly software for real-time data collection and analysis. In one embodiment, the improvement to the current technology, including improvements to computer hardware and software, provided by this system was benchmarked for accuracy and repeatability and tested clinically at the National Institutes of Health (NIH) clinical center with 42 craniofacial deformity patients in one embodiment. The data collected from the system was analyzed to validate system performance for research and clinical use.

These tests shows the system's improvement over the current technology and confirmed the system's high accuracy. In one embodiment, a 70 mm standard test bar was used, and the measured average static error is 0.03 mm, and dynamic error is 0.16 mm. The system demonstrated high repeatability, with a standard deviation of 0.02 mm among five repeated tests. Bite force sensor calibration with 5 masses (˜1 kg each) produced a linear force/voltage relationship, with R2 value of 0.998. Data was able to be collected efficiently in less than 15 minutes per session, proving the effectiveness of this system over the current technology including data collection in dental clinics.

This system advances both TMD research and clinical practice. With its demonstrated accuracy and user-friendliness, the system advances TMD research, particularly in deep phenotyping using machine learning to explore the relationships between TMJ structure, function, and pain. It also enables effective diagnosis and personalized treatment strategies by providing healthcare providers with comprehensive functional assessment results. The system can be used for expanding in-clinic analysis capabilities and performing large multicenter data collection to improve our understanding of TMD. Additionally, this system can be applied in other musculoskeletal disorders.

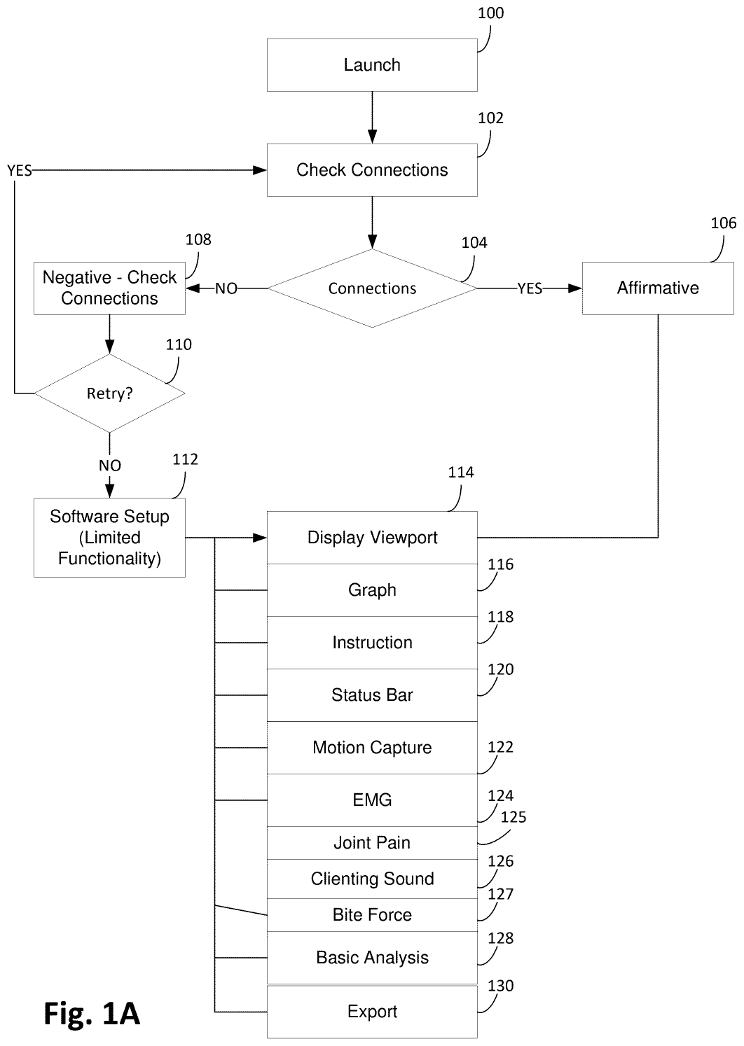

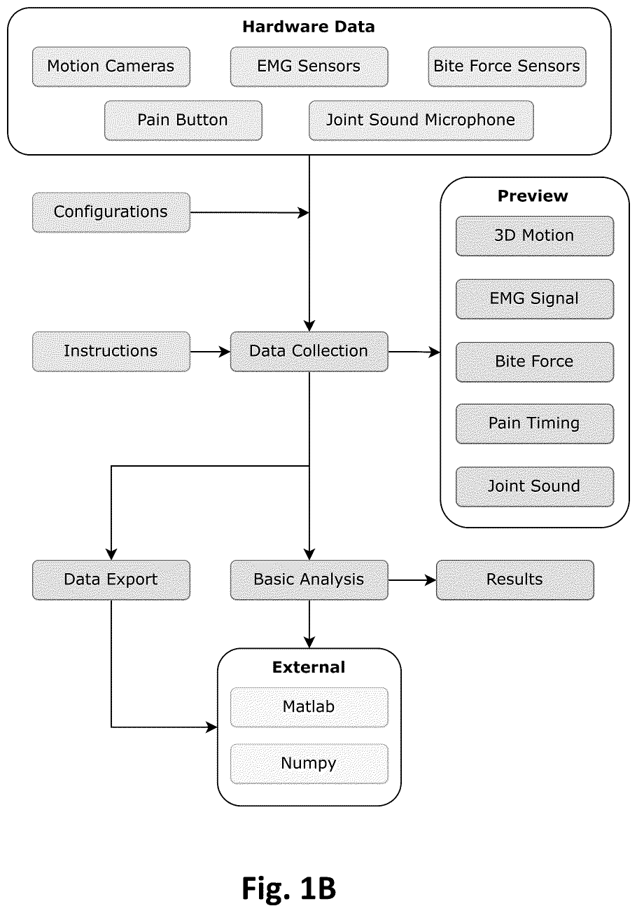

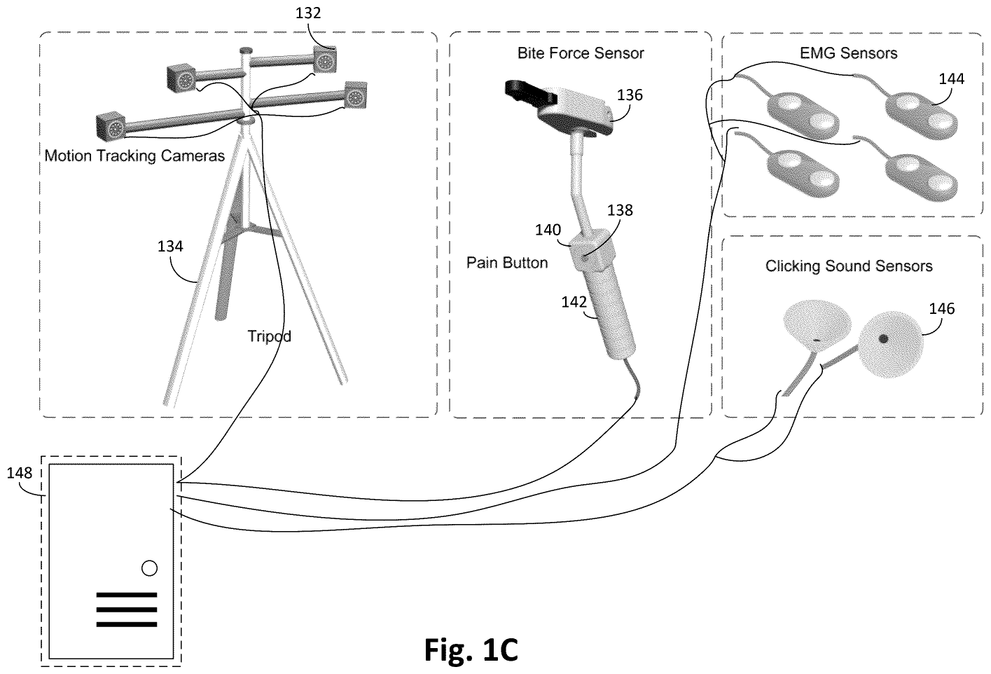

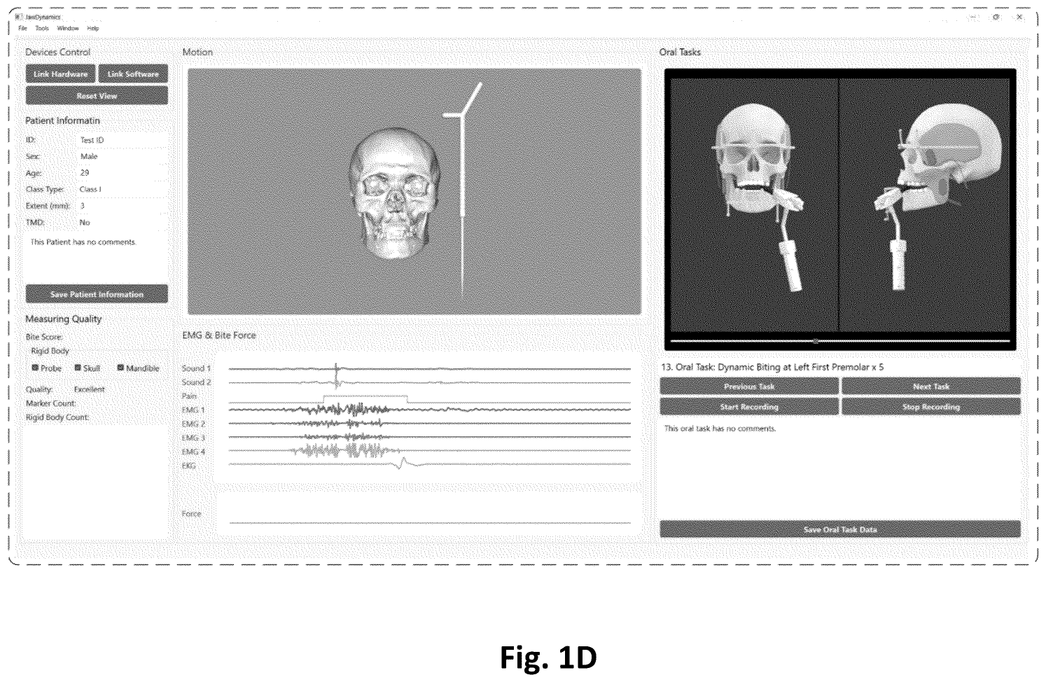

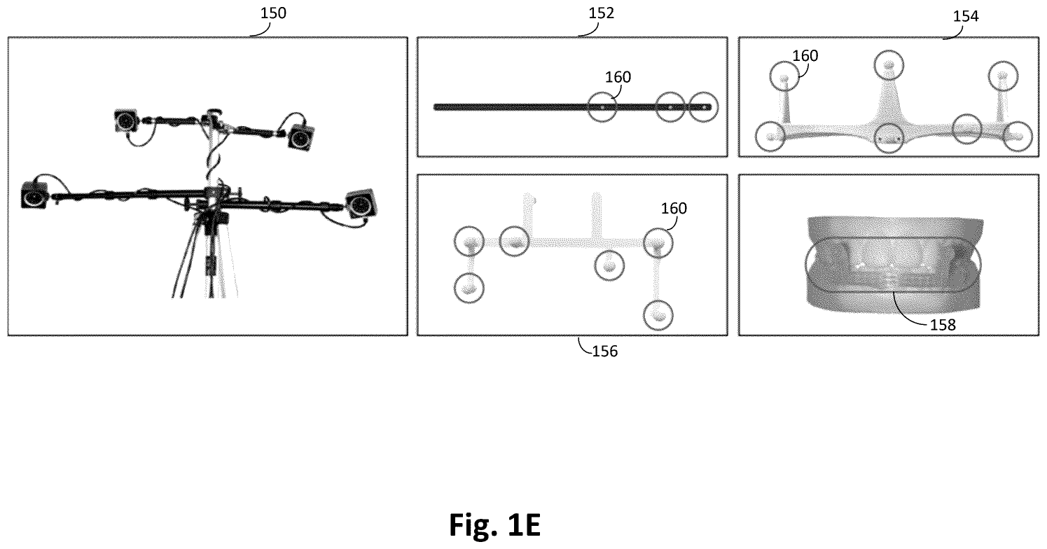

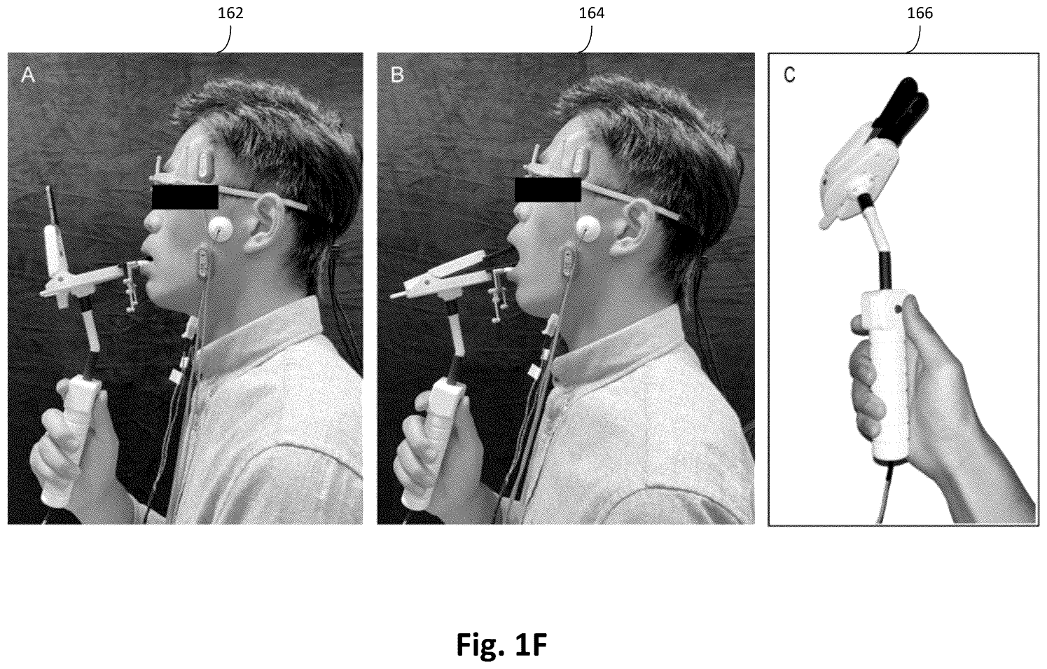

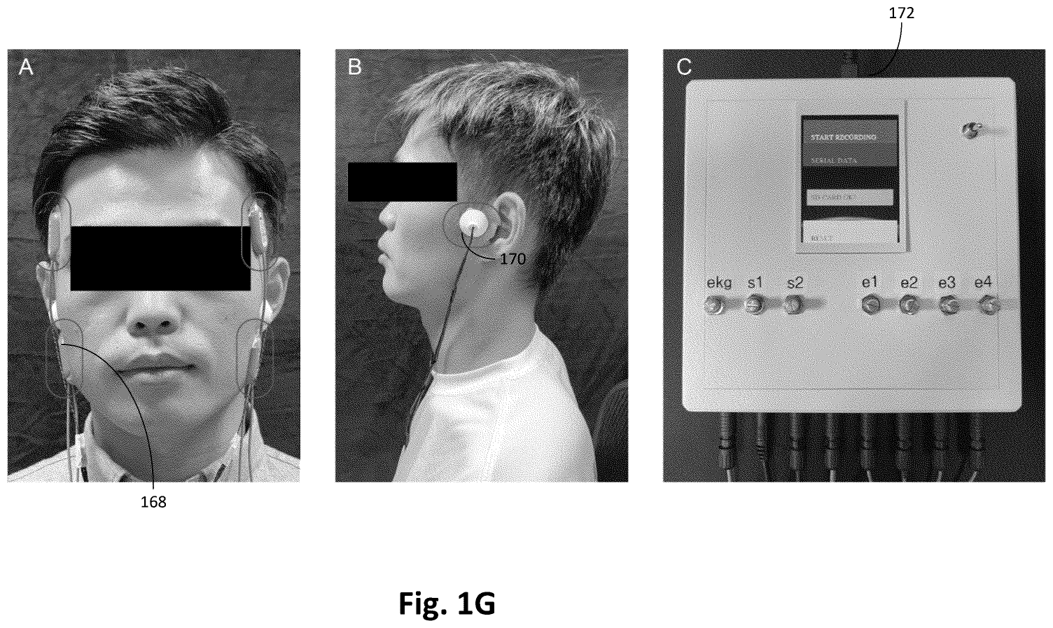

Referring to FIG. 1, the system can include computer readable instructions operatively associated with hardware and adapted for the collection and analysis of jaw kinematics, muscle activation, and bite force data. With built-in assessment modes and graphical step-by-step instructions, the system guides users through the precise measurement and data collection process, ensuring accuracy and efficiency in data collection. Data collected with the system can be used alone or for building state-of-the-art multiscale biomechanical models to analyze the mechanical and biological environment inside the jaw and jaw joint. These functions make this system a valuable tool and improvement of a computer system for studying jaw disease etiology and performing patient diagnosis.

Referring to FIG. 1, the system functionality provided by the computer readable instructions is illustrated. At 100, one embodiment of the software can be launched on a desktop or select it from your computer's Start menu. This action opens the software's main dashboard, allowing you to check with hardware connection or perform simple analysis. In one embodiment, the software automatically checks for hardware connections at 102. Should all necessary hardware be connected at 104, a message box at 106 will inform the user that the system is ready for data collection. Conversely, if any hardware component is not detected, the user will receive a notification at 108 detailing which hardware is missing and instructions to check the connection. Users have the option at 110 to retry connecting the hardware or to proceed with the software setup 112, albeit with limited functionality until all hardware components are successfully connected. The system can effectively check and establish connections with its corresponding hardware system. This feature ensures that all necessary devices are properly connected and communicate with the software. Users are guided through a straightforward process to check and confirm the connectivity of motion sensors, EMG electrodes, and bite force meters. Successful hardware is used for accurate data collection and analysis.

In one embodiment, menus of the system can users with easy access to all of the software's features and settings. Menus are grouped into different groups including file, motion, EMG, bite force, instruction, help, etc. A toolbar can complement the menu bar by offering quick, one-click access to the most frequently used functions and settings within the software. Since each user may have their own frequently used functions, the toolbar can be designed to be customizable.

The system can display a 3D motion viewport 114 that can serve as an advanced visualization tool, offering a dynamic 3D representation of jaw movements. Should users have prior access to a subject's skull information, they can seamlessly register this data to visualize real-time jaw motion tailored to the specific individual. In the absence of such personalized data, the system can use a generic model to facilitate the previewing of jaw movements.

In one embodiment, the EMG and bite force graph 116 can display real-time EMG and bite force data, enabling users to monitor muscle activity and biting strength instantly. For specific tasks, users can set target bite force levels or curves for subjects to follow. Instruction panel 118 can offer offers step-by-step instructions for each oral task, incorporating figures, text, and videos for comprehensive guidance. It also includes a timing function for tasks requiring specific durations. This panel can be detached and moved to a separate screen, allowing subjects to easily view the instructions during tasks, facilitating clear communication and accurate task execution. Status bar 120 can display real-time status information and notifications about the software's operations, including data collection progress, hardware connection status, and any alerts or messages relevant to the user's current activities.

In one embodiment, the hardware setup is verified, and users can proceed to motion capture functionality 122. This feature allows for the tracking of jaw movements, capturing detailed kinematics that are essential for understanding mandibular function and diagnosing disorders. The motion capture data, enriched with 3D visualization of jaw movement, offers invaluable insights into the mechanics of jaw movements. EMG measurement 124 can provide real-time monitoring of muscle activation patterns. This functionality is used for examining the muscular activities involved in jaw movements and identifying any dysfunctions or asymmetries in muscle activation that could affect oral health. Pain joint 125 can be determined and in one embodiment detected by the user activating a pain indicator such as a switch. Microphones can be used to detect clicking sounds at 126 that are produced when the joint is in motion (e.g., activated). Bite force measurement 127 functionality can quantify the force exerted during biting tasks. This information is used for assessing the strength and control capacity of the jaw, as well as for evaluating the impact of treatments aimed at improving bite force. The system also provides functionality of assigning target bite force curves for the subject.

The system can include basic analysis tools 128 that can allow users to immediately process and interpret motion, EMG, and bite force data within the system. These tools are designed for quick, preliminary data evaluation, offering functionalities like statistical summaries, trend identification, and comparisons across sessions or between subjects. The system also allows users to export at 130 collected data for extended analysis beyond the software's built-in analysis features. This functionality can support interoperability with programming languages such as MATLAB or Python, enabling researchers and professionals to apply custom analyses, build multiscale biomechanical models, or integrate data into broader research projects.

Referring to FIG. 1B, in one embodiment, the system computer readable instructions allow a user to start the system and method by launching the software either through the desktop icon or from the Start menu. Upon opening, the software's main dashboard is displayed. After launch, the system checks for connected hardware. A message box informs the user of the system's readiness or indicates missing components. Depending on the hardware check, users can retry the connection or proceed with available functionalities. Users can adjust data collection configurations for the data collection session via the menu bar or toolbar. For example, for motion capture, users can set subject skull geometry or choose to use generic skull model; for bite force measurement, users can set target levels or curves if needed. The users can guide the subject so that the subject follows step-by-step instructions displayed in the instruction panel for each selected task. As data is collected, users can monitor progress and quality through the 3D motion viewport and the EMG and bite force graph. After data collection, users can immediately analyze the data using basic analysis tools for preliminary insights. The software can provide statistical summaries, trend analysis, and session comparisons directly within the interface. For in-depth analysis, data can be exported to formats compatible with MATLAB or Python, allowing for extended research or clinical study. After saving or exporting data, users can close the session, ensuring all data is securely stored or ready for external analysis. The user can also actuate a pain indicator (e.g., button) that can represent that the user is experiences pain.

When visualizing the data, cone-beam computed tomography (CBCT) can provide a view of the relevant anatomy. Measurements of relevant bone geometry, such as mandible length and condyle shape, can be provided by CBCT scans and tracking. Further, the relative length of the mandible may be determined in this way. The CBCT co-registration process may involve capturing three-dimensional images of the subject's skull and jaw structures using cone-beam computed tomography imaging. The motion tracking markers may be visible in both the CBCT images and the motion capture system, allowing for precise spatial registration between the anatomical structures and the tracked motion data. In some embodiments, the system may overlay the detected motion trajectories directly onto the three-dimensional CBCT images, providing real-time visualization of jaw movement relative to the actual bone geometry. This co-registration process may enable accurate mapping of mandibular and skull motion to specific anatomical landmarks, facilitating precise biomechanical analysis.

Referring to FIG. 1C, aspects of the hardware of the system are shown. Motion tracking cameras 132 can be mounted on a stand 134 such as a tripod allowing the cameras to track the motion of the subject. A bit force sensor 136 can be used to measure bite force. A pain actuator 138 (e.g. button) can be carried by the bite force sensor so that when the subject bites and experiences pain, the system can record the bite force when pain occurs as well as instances of pain. The pain button can be separate from the bite force sensor and does not have to be integrated into the housing 140 or connected to a handle 142. The system can record data from EMG sensors 144 and from audio sensors 146. These components can be connected to a computerized controller 148.

In operation, a screen that is provided by computer readable instructions and hardware (e.g. computerized system and display) is shown as FIG. 1D.

The system can include compact hardware components and a user-friendly software component. The hardware can include a motion capture component for tracking mandible and skull motion, a bite force recording component with pain button for recording static and dynamic bite force as well as the instance when pain occurs, a skin surface EMG component for recording masticatory muscle activation a real-time recording of a component for capturing joint clicking sounds. The software component of our system streamlines the data collection and analysis process with modules for real-time data preview and recording as well as step-by-step functional assessment instructions (FIG. 1D).

Referring to FIG. 1E, the hardware components for jaw motion capture, including motion capture cameras 150 and mounting pod, calibration wand 152, skull marker bracket 154, and mandible piece bracket 156 for attaching to the buccal surface of the anterior mandibular teeth arch 158.

In operation, the motion capture component tracks the movement of retroreflective motion tracking markers 160 that can be 5 mm diameter in one embodiment and can be mounted on rigid frames, which can be later registered to geometries obtained from CBCT. This process enables the calculation of motion trajectories for any point on the mandible or skull. This component can include four high-speed infrared cameras mounted to a tripod, allowing for accurately tracking passive markers in dental clinic. Each camera can be positioned approximately 1 meter away from the subject, leaving enough space for the examiner to move about while maintaining high motion tracking accuracy. In one embodiment, the use and placement of four cameras ensures that multiple cameras capture each marker at any given time point, providing robust data capture. The camera mounts can be arranged so that they are not on the same line, minimizing motion capture errors. The motion capture cameras are connected to a data hub, which is connected to the data recording computer through Ethernet port, ensuring fast and reliable data transfer. Before motion data collection, the cameras are calibrated using a custom-made compact calibration wand 152 to determine the coordinates of each camera. The calibration procedures for motion tracking cameras may involve a multi-step process to establish the spatial relationship between cameras and define the measurement coordinate system. In some embodiments, the calibration wand may contain precisely positioned retroreflective markers at known distances, which are moved through the capture volume in specific patterns. The calibration software may automatically detect marker positions and calculate camera parameters including intrinsic properties such as focal length and lens distortion, as well as extrinsic properties such as camera position and orientation. The calibration process may also establish a global coordinate system that can be registered to CBCT imaging coordinates. Quality metrics may be provided during calibration to ensure measurement accuracy meets specified tolerances before data collection begins. After calibration, the data from multiple motion capture cameras can be used to reconstruct the 3D trajectory coordinates of reflective markers. Considering the complex translation and rotation involved in TMJ motion, at least three unique rotationally asymmetric configurations of markers per body may be needed to capture the 3D rigid body motion of the mandible and skull accurately. To ensure accuracy, a skull marker bracket can be used that include seven reflective markers to capture skull motion and a mandible marker bracket with six reflective markers to capture mandibular motion. The skull marker bracket 154 can be tightly attached to the patient's head during both motion capture and CBCT imaging, enabling the registration of motion captured by the camera system to the CBCT imaging coordinates. The mandible marker bracket 156 can be connected to the teeth via a disposable mandible bracket 158 which is attached to the buccal surface of the anterior mandibular arch of the patients using a 2 part bis-acrylic polymer.

Referring to FIG. 1F, the bite force and pain recording hardware is shown. Hardware components for bite force and pain recording, including a static bite force measurement part 162, dynamic bite force measurement part 164, and pain button 166. In one embodiment, to record static and dynamic bite force, a bite force device was designed. In static bite mode the patient bites on a force sensor covered by a rubberized plastic bite surface, 1 mm in one embodiment. This device measures static bite force at a nearly closed mouth position. In dynamic bite mode the patient bites on two hinged bite surfaces, one of which contains a force sensor, loaded via a torsional spring, allowing for the measurement of bite force during mandibular motion under load at 164. Additionally, a button is located on the handle of the bite force device allowing the subject to send a signal which is recorded whenever pain is felt. The pain signal is recorded in synchrony with all the other data such as those captured with the motion capture system and EMG sensors. This apparatus allows for the retrospective analysis of mandibular position, masticatory muscle activation, and joint force at the moment of pain. By correlating these data points, clinicians can gain valuable insights into the causes of the pain, facilitating targeted treatment.

Referring to FIG. 1G, hardware components are shown for the EMG recording and hardware component for TMJ clicking sound recording, including EMG sensors 168, high-sensitivity microphone 170 for recording TMJ clicking sound, and data collection hub 172. A multichannel skin surface EMG system can be used to record EMG signals. SX230 wired EMG sensors can be placed on the masticatory muscles (left and right masseter and temporalis). To record TMJ clicking sounds, a highly sensitive MEMS microphone (e.g., CMM-3729, CUI Devices) within an acoustic chamber can be placed directly TMJ area of the subject. Analog signals from the bite force device, pain button, EMG sensors and TMJ microphones are sent to the data collection computer through a custom data collection hub which, in one embodiment, amplifies and records sensor data at 5000 Hz. The system may provide data synchronization capabilities at high sampling rates, with the ability to synchronize multiple data streams at sampling rates of 1000 Hz or higher. In some embodiments, the data collection hub may sample all sensor inputs simultaneously, ensuring temporal alignment between motion capture data, EMG signals, bite force measurements, pain indicators, and joint sound recordings. This high-frequency synchronization may enable precise correlation of biomechanical events across different measurement modalities.

The real-time data preview and recording module is an aspect of the software component, offering a unified interface for seamlessly previewing and collecting various data types. The data preview module allows real-time preview of TMJ movement, muscle activation, bite force, clicking sound and pain. If the subject has segmented CBCT data available, the software can provide real-time motion visualization using the subject's actual skull geometry. In cases where segmented CBCT data is not available, a generic skull geometry can be used instead. This ensures that users, including healthcare providers, can visualize the motion capture data accurately, allowing them to verify the motion and detect any potential errors during the capture process. The real-time preview and visualization capabilities may include dynamic three-dimensional rendering of jaw movement overlaid on anatomical models. In some embodiments, the system may provide real-time feedback through color-coded displays indicating data quality, sensor connectivity status, and measurement accuracy. The visualization interface may allow users to adjust viewing angles, zoom levels, and display parameters during data collection. Additionally, real-time graphs and waveforms may be displayed simultaneously, showing EMG activity, bite force curves, and motion trajectories as they are being recorded. Additionally, other collected data are displayed on the screen, enabling the user to gain a comprehensive understanding of the patient. This real-time feedback is crucial for ensuring that the data collected is precise and reliable, as the integrated preview capability allows users to see all relevant data streams simultaneously, providing a comprehensive understanding of the subject's TMJ function in real-time.

Features like notetaking and data export further enhance the system's value and efficiency. The note-taking function allows users to document observations and insights directly within the system, capturing critical details during the functional assessment. For further analysis, such as computational modeling to calculate joint force, stress, and nutrient availability, the ability to export data for future use can be advantageous. The data export capability assists in ensuring that all relevant information, including motion capture, EMG signals, bite force measurements, and surgeon notes, is readily available for subsequent analysis.

The system setup and data collection instruction module are designed to provide comprehensive guidance and reminders for users such as clinical staff and subjects (e.g., patients) during the data collection process. This module can display step-by-step instructions and important reminders for the user, ensuring that the system is set up correctly and that data collection protocols are followed accurately. Additionally, it provides clear instructions for subjects, helping them understand the tasks they need to perform. To enhance understanding and compliance, the module can utilize multimedia elements including videos, audio, pictures, and text. These multimedia resources ensure that users, regardless of their familiarity with the system, can easily follow the instructions, resulting in a smooth and efficient data collection process.

In testing one embodiment of the system, the biomechanical functional assessment system was set up in a dental examination room at NIH clinical center. 42 patients with craniofacial deformities were assessed. The clinical staff received a brief 30-min training before starting to take measurements. The user training requirements and clinical workflow integration may involve structured training protocols designed for healthcare professionals with varying levels of technical expertise. In some embodiments, the training program may include hands-on instruction covering system setup, calibration procedures, patient preparation, data collection protocols, and basic troubleshooting. The training may be delivered through multiple modalities including written manuals, video tutorials, and supervised practice sessions. Clinical workflow integration may involve adapting the system to fit within existing appointment scheduling, patient flow, and documentation requirements. The system may be designed to integrate with electronic health records and existing dental practice management software. Training materials may include step-by-step checklists, quick reference guides, and troubleshooting flowcharts to support clinical staff during routine use. The oral tasks performed are listed in Table 1 and included kinematic tasks such as maximum open-close, medial-lateral movement, and anterior-posterior movement, as well as loaded tasks like static and dynamic biting at different teeth. The duration of a full functional assessment for each patient was documented. Collected data, including motion capture, EMG signals, and bite force measurements were exported to a subject-specific model to calculate parameters such as masticatory muscle force, TMJ joint force, disc stress, and strain.

TABLE 1
Oral tasks performed in NIH clinical center.

| Step | Oral Task |
|---|---|
| 1 | Maximum Open and Close × 5 |
| 2 | Maximum Lateral Movement to The Left × 5 |
| 3 | Maximum Lateral Movement to The Right × 5 |
| 4 | Maximum Anterior Movement × 5 |
| 5 | Maximum Posterior Movement × 5 |
| 6 | Static Biting at the Left Frist Premolar |
| 7 | Static Biting at the Right Frist Premolar |
| 8 | Static Biting at the Central Incisor |
| 9 | Dynamic Biting at the Left Frist Premolar × 5 |
| 10 | Dynamic Biting at the Right Frist Premolar × 5 |
| 11 | Dynamic Biting at the Central Incisor × 5 |

Across five reset and recalibration trials, the system showed excellent repeatability in static accuracy (Table 2), with minimal variation in the measured test bar lengths. The motion capture system demonstrated an average static deviation of 0.03 mm, corresponding to a percentage error of 0.04% relative to the test bar's 70 mm length. The standard deviation of the measured lengths was 0.02 mm, underscoring the system's consistency. For dynamic tracking, the root mean square error (RMSE) was 0.16 mm, which translates to a percentage error of 0.23%. This confirms the system's capability in capturing movements with minimal deviation from the true path in both static and dynamic conditions. The system's accuracy specifications and benchmark testing results may demonstrate performance characteristics suitable for clinical and research applications. In some embodiments, the motion tracking accuracy may be validated using precision-manufactured test fixtures with known dimensions and movement patterns. Benchmark testing may include assessments of measurement repeatability, inter-operator reliability, and long-term stability. The system may achieve sub-millimeter accuracy in static measurements and maintain accuracy within acceptable tolerances during dynamic motion tracking. Temperature stability, electromagnetic interference resistance, and mechanical vibration tolerance may also be evaluated as part of comprehensive accuracy specifications. Bite force measurement results showed high linearity, with recorded forces closely matching the expected values from known weights. To assess the performance and linearity of the bite force sensors, known weights were applied, and the measured forces were compared with the expected values. The results demonstrated a strong linear relationship between the applied weights and the sensor output. The R2 value calculated from the linear regression was 0.998 indicating excellent linearity across the measurement range.

TABLE 2
Static length measurement for five reset and recalibration.

| | | | | | | Standard |
|---|---|---|---|---|---|---|
| Test 1 | Test 2 | Test 3 | Test 4 | Test 5 | Mean | Deviation |
| 69.99 | 69.94 | 70.01 | 69.98 | 69.95 | 69.96 | 0.02 |
| mm | mm | mm | mm | mm | mm | mm |

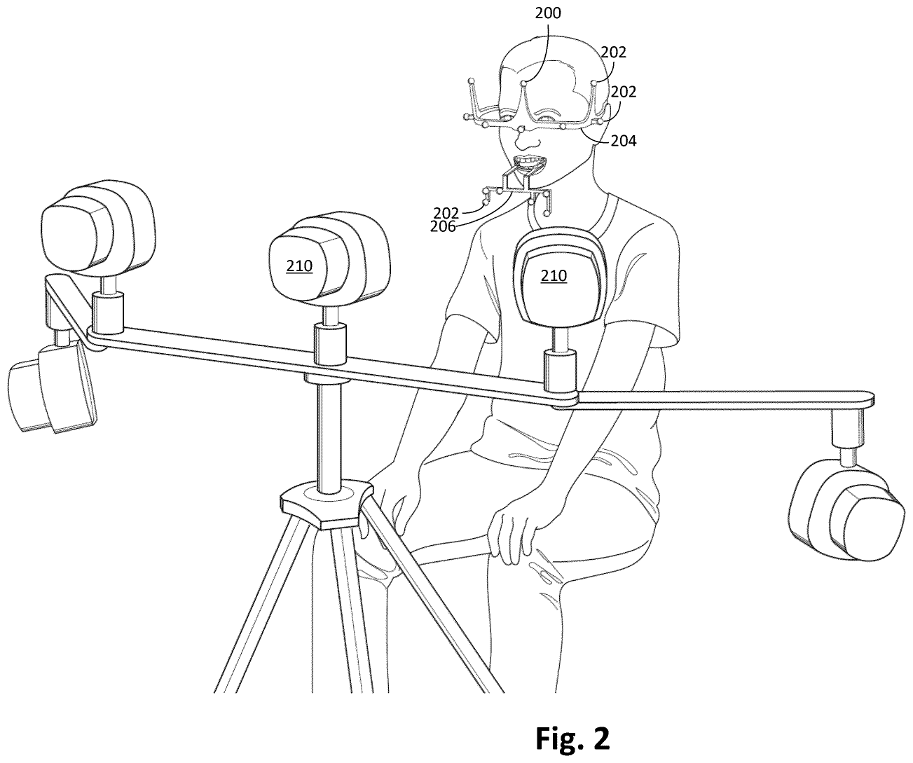

Referring to FIG. 2, kinematic measurements of movement of the jaw are provided by the system. Three-dimensional (3D) jaw motion tracking measures the motion of the mandible relative to the skull in three axes (x,y,z). In one embodiment, the motion tracking can track rigid body motion, including the rotation around x, y and z as well as translation along x, y and z. Aspects of the measurement can is performed using CBCT motion tracking markers 200 attached to a skull bracket 202 and a mandible bracket 206. The markers can be visible in CBCT and can be tracked by infrared cameras in one embodiment. Motion data is measured with a 3D motion tracking camera system 210.

Motion tracking markers 200 comprise retroreflective microspheres attached to the surface of a sphere containing CBCT contrast. The skull bracket 204 attaches to the head at the nasion and above the ears with a toggle strap to secure its position on the skull. In one embodiment, there are not fewer than seven (7) markers 200 on the skull bracket. As shown in the drawings, the markers are positioned asymmetrically from the center of the skull bracket.

Mandible bracket 206 is preferred to be formed in two parts, with a mouthpiece and an external part that may be disconnected from each other. The mouthpiece is formed as an arcuate member that conforms generally to the arc of the mandibular teeth and is attached to the anterior surface of the mandibular teeth (canines and incisors) with a nontoxic temporary adhesive, such as bis-acrylic polymer. The mouthpiece of the mandible bracket is constructed and arranged to attach to the lower mandibular teeth so as to not interfere with bite force measurement and to allow full closure of the patient's mouth while measuring motion. The mouthpiece is easily disconnected from the external part of the mandible bracket and may be disposable. The disposable components and attachment methods may include single-use mouthpieces that can be custom-fitted to individual patients for improved comfort and hygiene. In some embodiments, the bis-acrylic polymer adhesive may provide temporary but secure attachment that can be easily removed without damage to tooth enamel. The disposable mouthpiece may be pre-formed in various sizes to accommodate different patient anatomies, or may be moldable to create a custom fit. The attachment method may involve cleaning and drying the tooth surfaces, applying the adhesive material, and positioning the mouthpiece for optimal marker visibility and patient comfort. Removal procedures may include gentle warming or the use of specific solvents to dissolve the adhesive bond safely. In a preferred embodiment, small springs are used as pins to mount the mouthpiece to the mandible bracket for improved durability.

The motion tracking markers 200 are positioned at multiple spaced apart locations on the skull marker bracket 204 and on the mandible bracket 206. As shown in the drawings for this embodiment, the skull bracket has motion tracking markers positioned on each side of the skull and exterior to the skull, near the eyes of the patient, between the eyes of the patient, and above and between the eyes of the patient, and exterior to the skull and above the eyes.

The mandible bracket 206 has motion tracking markers 202 as shown in the drawings of this embodiment at multiple locations. These markers can be in a symmetrical design or can be in an asymmetrical design. The number of markers can vary. In one embodiment, the mandible has no fewer than six tracking markers. In one embodiment, the skull marker frame can include seven markers, and the mandible marker frame can include six markers. The markers can be positioned asymmetrically on the mandible bracket. The use of multiple motion tracking markers allow tracking and recording of position and orientation of the mandible and TMJ in three axes as the mandible is moved relative to the skull. In a preferred embodiment, no lines formed by any three (3) of the markers, including those on the skull bracket 204, are the same. The loss of one marker during the process of measuring jaw motion will still provide adequate resolution.

The infrared camera system records and displays in real time the relative 3D motion of the mandible to the skull during oral tasks such as biting and chewing. Motion data is co-registered with CBCT bone geometry using marker coordinates from both motion and CBCT data. These measurements and displays are preferably synchronized with simultaneous sEMG and bite force measurements, allowing for quantification and illustration of TMJ biomechanics, including mandible acceleration. The system may measure joint movement to less than 1 mm.

A microphone and recording equipment are used to record clicks and pops of the TMJ and the mandible moves relative to the maxilla. The position of TMJ when the pops and/or clicks can be traced when combining the microphone and motion tracking component.

The use of the skull bracket 204 in conjunction with the mandible bracket 206 provides accurate jaw motion information by the infrared camera system, even if the patient moves his or her head during the measurement process. The measurements are taken of the jaw movement relative to the position of the skull, rather than simply measuring the movement of the jaw that would distort the tracking measurements if the patient moved the skull during the measurement process. The use of an infrared cameras with the tracking markers according to the invention allows motion tracking to be performed with lights on in the room, which is believed to be preferred by both patients and technicians.

The tracking markers 202 are formed of radio opaque plastic and are preferred to be spherical in shape. The markers have an infrared reflective outer surface that provides parallel refection of infrared wavelengths back to the source, which in this case is the series of infrared cameras. In one embodiment, the coating is microscopic silica beads that are adhered to the plastic spheres. An inner surface is hydrophilic, and an outer surface is hydrophobic.

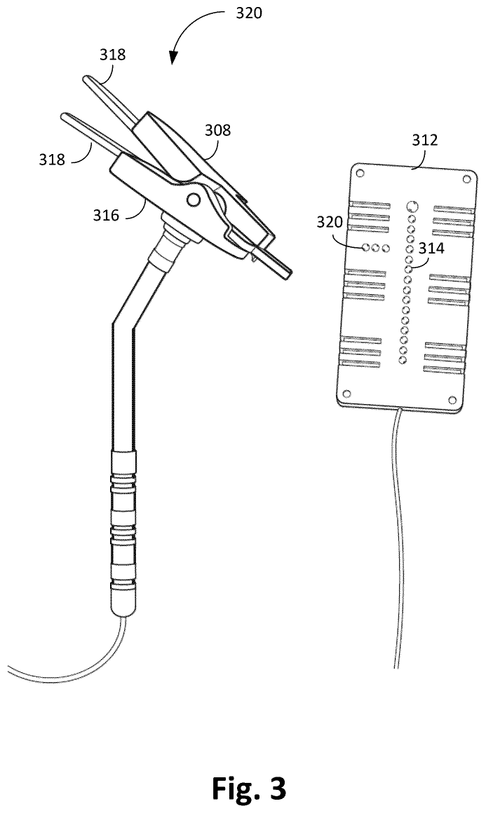

Referring to FIG. 3, as bite force device 300 and progressive meter 312 is shown. A top portion 308 can be hingably attached to a bottom portion 316 allowing the bit pads 318 to move apart and close relative to each other. When a user bites the bite pad, the force is measured and can be sent to the progressive meter 312. The progressive meter can include indicators 314 and 320 that can represent the force being applied to the bit force sensor. The bite force device may comprise a piezoresistive force sensor (preferably, having ˜100 N adjustable scale). The bite force device may be driven by an op-amp circuit to create an analog voltage signal which is input into an analog to digital converter. Disposable covers may be provided for the bite surface 318 of the bite force device. Operative bite force may be changed on the device in dynamic mode by increasing or decreasing spring biasing on the opposing paddles of the device.

Bite force visualization may be provided by progressive LEDs displayed on a connected visualization device 312. The visualization device displays real time bite force as an incremental bar graph of LEDs 314 and stoplight timer which indicates to the user when to start or stop applying biting force. The bite force device monitors bite force level, helps measure the bite force control capacity, and can improve quantification of the sEMG signals at various bite force levels.

The bite surface of the bite force device can be cantilevered to a loaded, spring biased, hinged bracket 316. Dynamic force is preferred to be recorded in concert with sEMG activity and 3D jaw motion tracking. Measurements of small muscle sEMG signals from the both left and right temporalis and masseter muscles are provided using fixed distance surface electrodes. The sEMG signals are amplified with an op-amp circuit to create analog voltage signals which are input into an analog to digital converter. The sEMG and force analog voltage signals are digitized with an analog to digital converter and serial data, such as 12-bit serial data, is transmitted to a computer via a microprocessor at a sampling rate of 1000 points per second, for example.

Some people may not be able to maintain bite force, which may be noted from static measurements. For example, a patient may be instructed to bite and hold at a predetermined level, such as 300 N, and for a time as shown by the LED indicators when the stoplight turns green. Failure to be able to hold for the required time is indicative of inadequate bite control capacity that may be related to TMJ and associated muscles. TMJ problems may require more use of the patient's muscles, resulting in earlier fatigue than would be experienced by a patient with normal TMJ function. Control deviation is measured by the system and recorded.

A patient may control the bite force visualization device 312 to indicate when pain begins as bite force is increased. In one embodiment, this can be accomplished with the patient depressing a button that indicates pain is felt. Determining when pain begins as bite force increases is an important indicator. The patient may use the bite force device at home and apart from the camera-based tracking system. The readings may be recorded, such as by using an SD card or other media or using near field communications connected to a computing device. Time to generate and hold bite force may be clinically important. Some people cannot generate or hold bite force. The stoplight timer 320, which may be a series of red, yellow and green LEDs on the bite force visualization device 312, indicate to the patient when to begin applying bite force. A delay in the patient's activity to apply bite force is indicative of avoidance psychology, which may be a valuable observation.

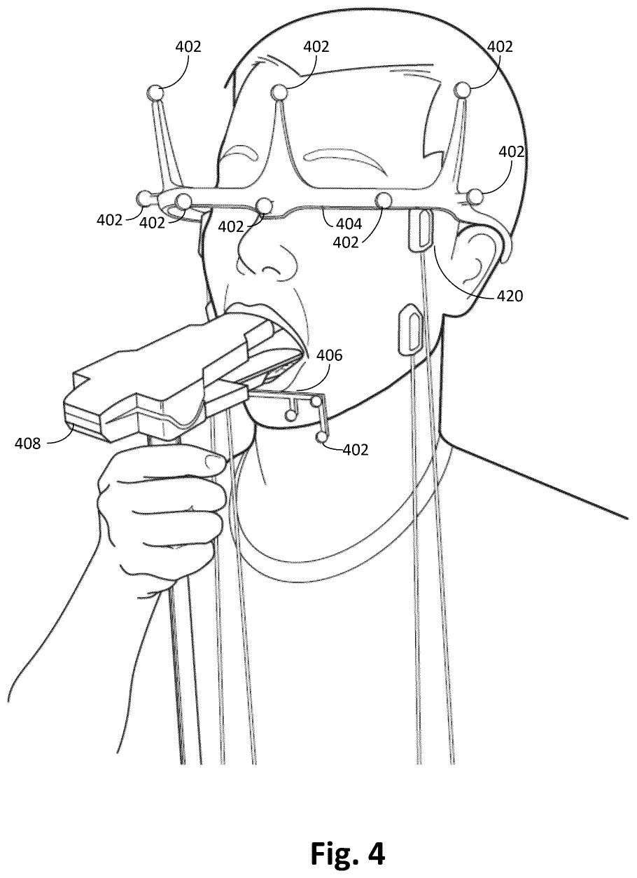

Referring to FIG. 4, EMG electrodes 420 are attached to each side of the patient's head (e.g., left and right masseter and temporalis muscles) near the TMJ. The EMG electrodes measure muscle activity which is displayed as a wave pattern. Various jaw movements result in a different wave patterns that are categorized and are indicative of jaw problems. Bite sEMG and motion tracking may be performed simultaneously. The signals of each may be synchronized so that the mandible position is known as the jaw speed and muscle activity is also known. The use of a pain button on the bite force device can be in static and dynamic mode and can indicate jaw position, location, speed and some combination where pain occurs.

sEMG measurements work together with bite force measurements. A bite force device 408 may be used in static or dynamic bite mode. In both static and dynamic mode, bite force is preferred to be recorded in concert with sEMG activity and 3D jaw motion tracking. FIG. 4. Muscles are used differently for static biting and dynamic biting, and it may be desirable to acquire both static and dynamic readings of bite force.

In one embodiment, each of the following can have a different sEMG signal pattern output: rhythmic clenches; molar tapping; static incisor bite on saliva ejector; Rhythmic incisor bites on saliva ejector; grind on right canine; grind on left canine; dynamic jaw play (right-left movement); smile; right side tough meat chewing; left side tough meat chewing; right side gum chewing; left side gum chewing; dynamic right molar bites on saliva ejector; clenching extremely hard. In one case, the observed pattern can be compared with a pattern recognition standard to identify one or more deviations from the standard.

The combined CBCT co-registered 3D jaw motion tracking, sEMG, bite force measurements, pain and joint clicking sounds to provide an improved paradigm for the quantification of 3D TMJ biomechanics and provide input for novel software and computational modeling. The system quantifies baseline masticatory function and aids clinicians in diagnosing TMJ disorders and improving treatment efficacy. By determining bite force and muscle activity, TMJ force at the condyle and disc may be determined through an inverse dynamics model.

One embodiment of this biomechanical functional system was successfully implemented in dental examination rooms at NIH clinical center. The system's compact design allows it to fit into two small suitcases (˜22×14×9 inches) with a total weight of less than 30 kg, making it easily portable. In one embodiment, it can be about 10 kg. The dental examination room at NIH clinical center measures 3.2×3.0 meters and all necessary equipment fit comfortably within the available space. The portable and compact design features may include modular components that can be quickly assembled and disassembled for transport. In some embodiments, the motion tracking cameras may be mounted on lightweight, collapsible tripods that can be folded for storage. The data collection hub and computerized controller may be housed in ruggedized cases designed for clinical transport. The system's portability may enable deployment in various clinical settings, from dental offices to research facilities, without requiring permanent installation or significant space modifications. Data collection was efficiently conducted by a clinical assistant or surgeon after a 30-min training period. The system setup and calibration took 15 minutes, and each data collection session took approximately 15 minutes to complete, ensuring that the process was efficient and minimally disruptive to clinical workflows. The ease of setup and quick training time highlight the system's practicality for routine clinical use, demonstrating its potential for widespread adoption in dental clinics.

The collected results were exported to third party software (e.g., ArtiSynth) for joint load and muscle force analysis using the rigid body dynamic and finite element model. This advanced patient-specific model imports subject-specific morphology from CBCT data and kinematics, muscle activation, and bite force from our biomechanical functional system to calculate joint load, muscle forces, disc and capsule stress and strain. The computational modeling integration and export capabilities may include standardized data formats that facilitate seamless transfer to various analysis platforms. In some embodiments, the system may export motion data in formats compatible with biomechanical modeling software such as ArtiSynth, OpenSim, or MATLAB. The export functionality may include synchronized datasets containing temporal alignment of all measurement modalities, enabling comprehensive biomechanical analysis. Data export formats may include CSV files for numerical data, XML files for structured metadata, and specialized formats for three-dimensional motion trajectories. The system may also provide application programming interfaces (APIs) that allow direct integration with computational modeling workflows. Export capabilities may include patient-specific geometric models derived from CBCT data, kinematic parameters, muscle activation patterns, and force measurements, all properly scaled and calibrated for use in finite element analysis or multibody dynamics simulations. The computational analysis provided a detailed understanding of the mechanical environment within the TMJ, which could help to predict the effects of surgical interventions and other treatments on joint health.

The complex and multifactorial nature of the TMD system provides a comprehensive approach that considers multiple factors in both studying its etiology and making clinical diagnoses and treatment plans. Among these factors, structure, function and pain are critical, interrelated aspects that significantly influence TMD. While the wide availability of imaging tools such as CBCT and MRI has enabled dental clinics to study and diagnose structural abnormalities like joint degeneration, clinical assessments often overlook the functional aspects due to limitations in available instrumentation. Although some commercially available products can measure jaw movement, EMG, or bite force, they cannot simultaneously record pain, limiting their capacity to help trace the source of the pain.

Furthermore, conducting a full functional assessment typically requires using separate hardware and software from different vendors, leading to inefficiencies and prolonged assessment times. In clinical settings, ease of use and efficiency are advantageous for successful implementation of these systems. To address these challenges, this TMJ biomechanical functional assessment system provides an accurate, user-friendly, and integrated solution that enables recording of jaw movements, muscle activation, bite force, pain and clicking sound during various oral tasks. This system paves the way for understanding the relationships between TMJ structure, function, and pain, while also helping design accurate diagnoses and targeted treatment plans.

The benchmark test results confirmed the accuracy of this TMJ biomechanical functional assessment system, which is advantageous for effective TMJ functional assessment. Given that TMJ movements are inherently small and that the jaw tracking brackets can be attached to the teeth rather than the joint itself, this high level of accuracy ensures that the motion captured at the joint is accurate. The reliability of the EMG signals and bite force sensors further reinforces the accuracy of the entire system, which is critical not only for direct measurements and functional assessments but also for integrated computational modeling. In computational modeling, inaccuracies in functional assessment data can impact the calculation of muscle forces and joint loads, potentially leading to incorrect conclusions or suboptimal treatment plans. Therefore, the validated accuracy of our system is essential for generating reliable data that can be used confidently in both research and clinical settings.

Clinical testing results demonstrate that this TMJ biomechanical functional assessment system is easy to use, including when used in the dental clinic chairside application. The system's compact and portable design allows it to be conveniently set up in standard dental examination rooms, requiring minimal space and effort. During clinical testing at NIH clinical center, the system was highly rated by clinical staff for its user-friendly interface. The entire data collection process, including functional assessments during multiple oral tasks, was completed in approximately 15 minutes per session, demonstrating the system's capability to provide comprehensive TMJ assessments without causing disruption to clinical workflows. As oral research advances, more targeted functional assessments focused on a specific subset of oral tasks could further reduce assessment time.

This system is suitable for widespread adoption in dental clinics, offering a reliable and efficient tool for collecting extensive patient data to better understand TMJ etiology and improve the diagnosis and monitoring of TMD and TMJ etiology.

The TMJ functional assessment system has the potential to significantly advance both TMD research and clinical practice. In TMD research, this system can collect large amounts of high-quality functional data including jaw kinematics, muscle activation, bite force, joint clicking, and pain. Such functional data, combined with CBCT and MRI imaging data, supports advanced TMD etiology studies through deep phenotyping with explainable machine learning and multiscale computational modeling, offering critical insights into the relationship between TMJ structure, function, and pain. Clinically, the system's capacity to gather detailed patient TMJ functional data allows for more accurate and nuanced diagnoses, enabling healthcare providers to develop more personalized treatment plans.

In one embodiment, the system can provide advanced kinematic analysis and computational modeling without the need to export to external tools. This TMJ functional assessment system provides an accurate, user-friendly, and integrated solution for real-time TMJ functional assessment, effectively filling critical needs for functional assessment in both research and clinical practice. Benchmark testing confirmed the system's accuracy and reliability, while clinical trials demonstrated its practicality as a tool in dental settings. The system can be used to advance research on the relationship between TMJ structure, function, and pain, while also enhancing the clinical management of TMD. The system's ability to collect large amounts of multi-modal functional data assists with advancing TMD research, particularly in deep phenotyping with machine learning and the development of effective diagnosis and treatment strategies. This system can assist in the development of new standards in TMD diagnosis and treatment, ultimately leading to a deeper understanding of TMD and improved patient outcomes. This system can be used for improving the understanding the relationship between structure, function, and pain and can be extended beyond the TMJ to other musculoskeletal disorders, potentially improving overall musculoskeletal health.

The system may find practical application across multiple clinical and research settings, where its integrated approach to TMJ assessment can enhance patient care and scientific understanding. In dental clinics, the system may enable practitioners to conduct comprehensive TMJ evaluations in approximately 15 minutes, providing simultaneous measurement of jaw kinematics, muscle activation, bite force, and pain responses during routine examinations. The portable design allows the system to be transported between clinical locations, making it suitable for use in specialized TMJ clinics, orthodontic practices, and oral surgery centers. In research environments, the system may facilitate large-scale data collection for TMD studies, enabling researchers to gather synchronized multi-modal datasets that can be analyzed using machine learning approaches to identify patterns and relationships between structure, function, and pain. The system's ability to export data to computational modeling platforms may support the development of patient-specific treatment plans, allowing clinicians to predict the outcomes of surgical interventions or orthodontic treatments before implementation. Additionally, the system may serve as a monitoring tool for tracking treatment progress over time, providing objective measurements to assess the effectiveness of various therapeutic approaches and enabling evidence-based adjustments to treatment protocols.

One or more different inventions may be described in the present application. Further, for one or more of the invention(s) described herein, numerous embodiments may be described in this patent application, and are presented for illustrative purposes only. The embodiments described are not intended to be limiting in any sense. One or more of the invention(s) may be widely applicable to numerous embodiments, as is readily apparent from the disclosure. These embodiments are described in sufficient detail to enable those skilled in the art to practice one or more of the invention(s), and it is to be understood that other embodiments may be utilized and that structural, logical, software, electrical and other changes may be made without departing from the scope of the one or more of the invention(s). Accordingly, those skilled in the art will recognize that the one or more of the invention(s) may be practiced with various modifications and alterations. Particular features of one or more of the invention(s) may be described with reference to one or more particular embodiments or figures that form a part of the present disclosure, and in which are shown, by way of illustration, specific embodiments of one or more of the invention(s). It should be understood, however, that such features are not limited to usage in the one or more particular embodiments or figures with reference to which they are described. The present disclosure is neither a literal description of all embodiments of one or more of the invention(s) nor a listing of features of one or more of the invention(s) that must be present in all embodiments.

Headings of sections provided in this patent application and the title of this patent application are for convenience only, and are not to be taken as limiting the disclosure in any way.

It is understood that the above descriptions and illustrations are intended to be illustrative and not restrictive. It is to be understood that changes and variations may be made without departing from the spirit or scope of the following claims. Other embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventor did not consider such subject matter to be part of the disclosed inventive subject matter.

What is claimed is:

1. A system for quantification of human temporomandibular joint mechanics and oral function, comprising:
a skull motion tracking bracket configured to be carried by a subject and having a plurality of motion tracking markers positioned asymmetrically thereon;
a mandible motion tracking bracket configured to be carried by the subject and having a plurality of motion tracking markers positioned thereon;
a set of motion detecting cameras adapted to detect and record data representing movement of the motion tracking markers on the skull motion tracking bracket and the mandible motion tracking bracket; and
a computerized controller adapted to receive the motion data from the set of motion detecting cameras, receive a set of three-dimensional images of the subject, co-register the motion data with the three-dimensional images, and display an overlay of the detected motion with the three-dimensional images to a user.

2. The system of claim 1, further comprising a set of electromyography sensors carried by the subject and adapted to detect and record facial muscle activity data and transmit the data to the computerized controller.

3. The system of claim 2, further comprising a set of audio sensors carried by the subject and adapted to detect and record sound data associated with jaw movement and transmit the data to the computerized controller.

4. The system of claim 3, further comprising a bite force sensor adapted to detect and record bite force data of the subject and transmit the bite force data to the computerized controller.

5. The system of claim 4, further comprising a pain actuator adapted to be activated by the subject to indicate pain and transmit pain data to the computerized controller.

6. The system of claim 1, wherein the skull motion tracking bracket comprises at least seven motion tracking markers and the mandible motion tracking bracket comprises at least six motion tracking markers.

7. The system of claim 6, wherein the motion tracking markers comprise retroreflective microspheres attached to a surface of a sphere containing cone-beam computed tomography contrast material.

8. A system for quantification of human temporomandibular joint mechanics and oral function, comprising:
a skull motion tracking bracket configured to be carried by a subject and having motion tracking markers positioned thereon;
a mandible motion tracking bracket configured to be carried by the subject and having motion tracking markers positioned thereon;
a set of motion detecting cameras adapted to detect and record motion data from the motion tracking markers;
a set of electromyography sensors configured to be carried by the subject and adapted to detect and record facial muscle activity data;
a set of audio sensors configured to be carried by the subject and adapted to detect and record sound data associated with jaw movement;
a bite force sensor adapted to detect and record bite force data of the subject; and,
a computerized controller adapted to receive the motion data, the facial muscle activity data, the sound data, and the bite force data and synchronize the received data for analysis.

9. The system of claim 8 including a pain actuator configured to detect a pain signal from the subject and wherein the a computerized controller adapted to receive the pain signal and synchronize the received data for analysis.

10. The system of claim 8, wherein the skull motion tracking bracket comprises a plurality of motion tracking markers positioned asymmetrically thereon and the mandible motion tracking bracket comprises a plurality of motion tracking markers positioned thereon.

11. The system of claim 10, wherein the motion tracking markers comprise retroreflective microspheres attached to a surface of a sphere containing cone-beam computed tomography contrast material.

12. The system of claim 8, wherein the bite force sensor is configured to operate in both static and dynamic bite modes, the static mode measuring bite force at a nearly closed mouth position and the dynamic mode measuring bite force during mandibular motion under load.

13. The system of claim 12, wherein the bite force sensor comprises a piezoresistive force sensor within a flexible bite surface cover and is driven by an operational amplifier circuit to create an analog voltage signal.

14. The system of claim 8, wherein the set of electromyography sensors comprises surface electrodes configured to be placed on left and right masseter and temporalis muscles of the subject.

15. The system of claim 8, wherein the set of audio sensors comprises a highly sensitive MEMS microphone within an acoustic chamber configured to be placed directly on a temporomandibular joint area of the subject.

16. A temporomandibular joint assessment apparatus, comprising:
a skull tracking bracket having retroreflective motion tracking markers visible in cone-beam computed tomography imaging;
a mandible tracking bracket having retroreflective motion tracking markers and configured to attach to anterior mandibular teeth;
infrared motion tracking cameras positioned to capture three-dimensional motion of the retroreflective motion tracking markers;
a bite force measurement device having static and dynamic measurement modes and including a pain indicator button;
electromyography sensors configured for placement on masticatory muscles;
a microphone configured to detect temporomandibular joint clicking sounds; and
a data collection hub configured to synchronize and record data from the infrared motion tracking cameras, the bite force measurement device, the electromyography sensors, and the microphone at a sampling rate of at least 1000 Hz.

17. The temporomandibular joint assessment apparatus of claim 16, wherein the skull tracking bracket comprises a a plurality of seven retroreflective motion tracking markers positioned thereon.

18. The temporomandibular joint assessment apparatus of claim 16, wherein the mandible tracking bracket comprises a plurality of retroreflective motion tracking markers positioned thereon.

19. The temporomandibular joint assessment apparatus of claim 16, wherein the bite force measurement device comprises a piezoresistive force sensor and is driven by an operational amplifier circuit to create an analog voltage signal.

20. The temporomandibular joint assessment apparatus of claim 19, wherein the bite force measurement device in dynamic measurement mode comprises two hinged bite surfaces loaded via a torsional spring to allow measurement of bite force during mandibular motion under load.

21. A system for quantification of human temporomandibular joint mechanics and oral function, comprising:
a skull motion tracking bracket configured to be carried by a subject and having motion tracking markers positioned thereon;
a mandible motion tracking bracket configured to be carried by the subject and having motion tracking markers positioned thereon;
a set of motion detecting cameras adapted to detect and record motion data from the motion tracking markers;
a set of sensors taken from the group consisting of a electromyography sensor configured to be carried by the subject and adapted to detect and record facial muscle activity data, an audio sensors configured to be carried by the subject and adapted to detect and record sound data associated with jaw movement, a bite force sensor adapted to detect and record bite force data of the subject, pain actuator configured to detect a pain signal from the subject and any combination thereof; and,
a computerized controller adapted to receive a data from the set of sensors and synchronize the received data for subsequent analysis.

22. The system of claim 21, wherein the skull motion tracking bracket comprises a retroreflective motion tracking marker positioned thereon and the mandible motion tracking bracket comprises a retroreflective motion tracking markers positioned thereon.

23. The system of claim 22 wherein the motion tracking markers comprise retroreflective microspheres attached to a surface of a sphere containing cone-beam computed tomography contrast material.

24. The system of claim 21, wherein the bite force sensor is configured to operate in both static and dynamic bite modes, the static mode measuring bite force at a nearly closed mouth position and the dynamic mode measuring bite force during mandibular motion under load, and wherein the bite force sensor comprises a piezoresistive force sensor within a flexible bite surface cover driven by an operational amplifier circuit to create an analog voltage signal.

25. The system of claim 21, wherein the computerized controller is adapted to receive a set of three-dimensional cone-beam computed tomography images of the subject, co-register the motion data with the three-dimensional images using marker coordinates from both motion and imaging data, and display a real-time overlay of the detected motion with the three-dimensional images to provide visualization of jaw movement relative to actual bone geometry.

26. The system of claim 21, wherein the computerized controller is adapted to synchronize all received data at a sampling rate of at least 1000 Hz and export the synchronized data in formats compatible with biomechanical modeling software to enable calculation of joint forces, muscle forces, and stress distributions within temporomandibular joint structures.
